(19) United States
(12) Patent Application Publication
(10) Pub. No.: US 2009/0182260 A1
(43) Pub. Date: Jul. 16, 2009

(54) Dual-passage surgical device

(76) Inventor: Timothy R. Ronald, Clarence, NY (US)

Correspondence Address: Michael F. Scalise, 10755 Manor Wood, Clarence, NY, 14031, US

(21) Appl. No.: 12/221,112

(22) Filed: Jul. 31, 2008

Related U.S. Application Data
(62) Division of application No. 11/973,710, filed on Oct. 9, 2007, now abandoned.
(60) Provisional application No. 60/850,123, filed on Oct. 10, 2006.

(51) Int. Cl. A61B 10/02

(52) U.S. Cl. 604/22; 600/567

(57) ABSTRACT

A medical device for performing a surgical procedure is described. The medical device comprises a housing having a sidewall extending along a longitudinal axis. A primary passage in the housing extends from an open proximal housing end to a hypodermic needle at a distal end. A secondary passage extends from the open proximal housing end to a distal opening in the housing sidewall adjacent to the hypodermic needle. The secondary passage provides for administering a medicine to the surgical site through the distal opening in the housing sidewall without removing the needle device from the body. That way, the primary passage enables a surgeon to administer a first drug or medicine to a surgical site during a surgical procedure. If needed, the surgeon can administer a second drug or medicine to the surgical site through the secondary passage.

### CROSS-REFERENCE TO RELATED PATENT APPLICATIONS

This application is a divisional application of U.S. application Ser. No. 11/973,710, filed Oct. 9, 2007, which claims priority from U.S. provisional patent application Ser. No. 60/850,123, filed Oct. 10, 2006.

### BACKGROUND OF THE INVENTION

The present invention generally relates to a medical device for performing a surgical procedure. More particularly, the medical device comprises a dual-passage housing and a biopsy rod or a hypodermic needle, and the like. The housing is provided with two conduits or passages extending from a proximal end of the device held by the physician to a distal end that resides proximate to a surgical site. That way, the needle device enables a physician to operate on a surgical site from outside the body and should blood-coagulating medicine be needed at the surgical site, it may be readily delivered through the secondary passage without having to remove the needle from the body.

### SUMMARY OF THE INVENTION

A biopsy needle device for surgeons is described. The device comprises a housing having side-by-side passages. One passage houses a biopsy rod that extends to a window at the distal end of the housing. The biopsy rod has a cutting recess that is aligned with a biopsy window at the distal end of the housing. The cutting recess is rotated away from the biopsy window as the device is moved through the body to the surgical site. Then, the rod is rotated to a position where the cutting recess is aligned with the biopsy window. Natural body pressure causes tissue to move into the biopsy window and into the cutting recess. Further rotational movement of the biopsy rod cuts a sample of tissue for later analysis as to whether it is malignant or benign. The other housing passage is in fluid flow communication with an opening adjacent to the biopsy window. That way, should excessive bleeding or similar undesirable results occur during the biopsy, a medicine can be administered to the surgical site without withdrawing the biopsy needle device.

These and other objects of the present invention will become increasingly more apparent to those skilled in the art by reference to the following description and the appended drawing.

### BRIEF DESCRIPTION OF THE DRAWINGS

FIG. 1 is a perspective view of one embodiment of a needle device 10 according to the present invention comprising dual-passage housing 12 and a biopsy needle 14.

FIG. 1A is a perspective view of a biopsy rod 14.

FIG. 1B is a cross-sectional view along line 1B-1B of FIG. 1A.

FIG. 2 is a perspective view of an alternate embodiment of a dual-passage housing according to the present invention with a drug delivery tube 50 attached to a secondary passage 30 thereof.

FIG. 3 is an enlarged view of the distal end 20 of the dual-passage housing 12.

FIG. 4 is a cross-sectional view along line 4-4 of FIG. 3.

FIG. 5 is a cross-sectional view along line 5-5 of FIG. 3.

FIG. 6 is a perspective view of a second embodiment of a needle device 100 according to the present invention comprising a hypodermic needle 108.

The present invention will now be described in connection with preferred embodiments, however, it should be understood that there is no intent to limit the invention to the embodiments described. On the contrary, the intent is to cover all alternatives, modifications and equivalents as may be included within the spirit and scope of the invention as defined by the appended claims.

### DETAILED DESCRIPTION OF THE PREFERRED EMBODIMENTS

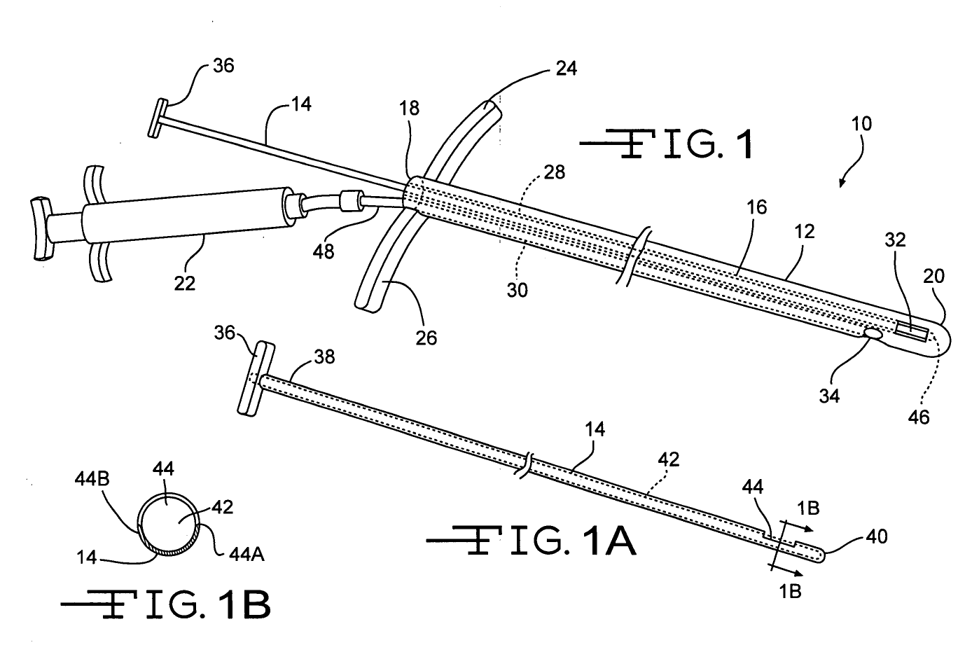

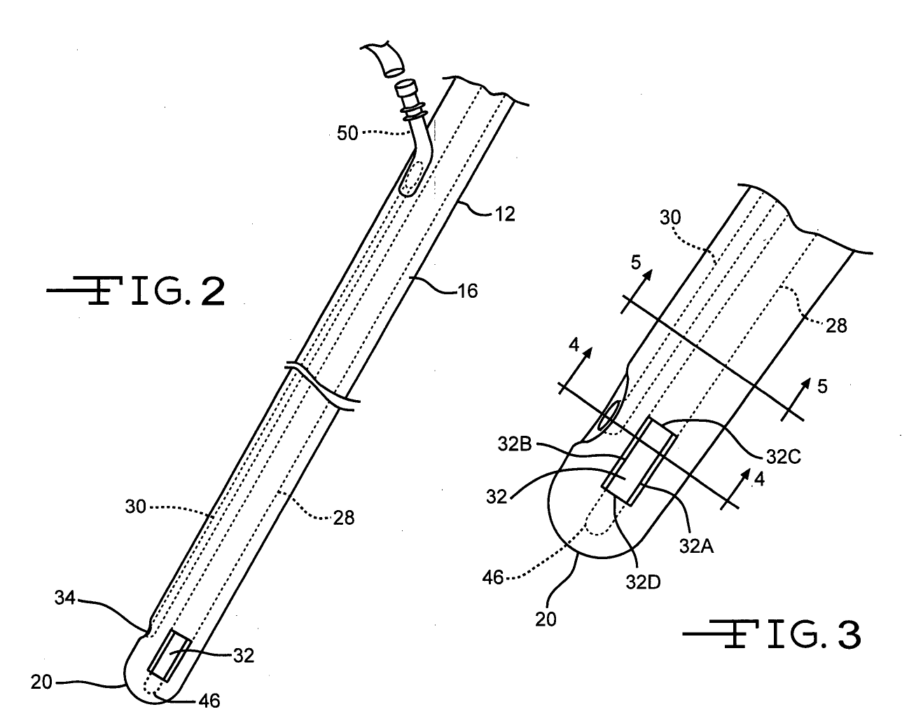

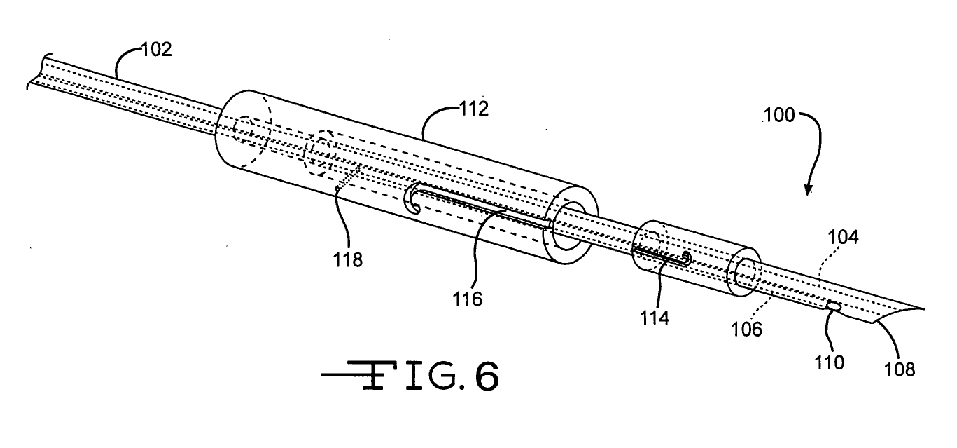

Referring now to the drawings, FIGS. 1, 1A, 1B and 2 to 5 illustrate one embodiment of a needle device 10 according to the present invention that is particularly adapted for performing biopsies. The needle device 10 comprises a dual-passage housing 12 and a biopsy rod 14. The dual-passage housing 12 is made of a metal such as titanium or stainless steel, or a polymeric material. In any event, the material of construction must be biocompatible and strong enough to withstand being moved into a body to perform a surgical procedure without suffering structural failure.

The housing 12 has a cylindrically-shaped sidewall 16 extending along a longitudinal axis thereof from a proximal end 18 to a closed distal end 20. The proximal housing end 18 is open so that the biopsy rod 14 can be moved into the housing and a drug delivery device 22, for example a syringe, can be connected to thereto. The terminus of the distal end 20 has a curved radius surface. The housing sidewall 16 supports oppositely extending wing-shaped members 24 and 26 that serve as a handle for the needle device 10. In use, the handle wings 24, 26 are grasped by the index and middle fingers as the needle device 10 is manipulated during a surgical procedure.

Side-by-side internal conduits 28 and 30 communicate between the open proximal end 18 and the distal end 20 of the housing 16. The first conduit 28 is a main passage that extends from the open end 18 along the longitudinal axis of the housing 12 to a rectangular-shaped window 32 adjacent to the distal end 20. The second conduit 30 is a secondary passage that extends from the open proximal end 18 along and parallel to the main passage 28 to a secondary opening 34 in the sidewall 16. The secondary opening 34 has a circular or oval shape and is at a proximal location with respect to the primary window 32. In other embodiments, the secondary opening is directly opposite the primary window 32 or at a distal location thereof, if desired.

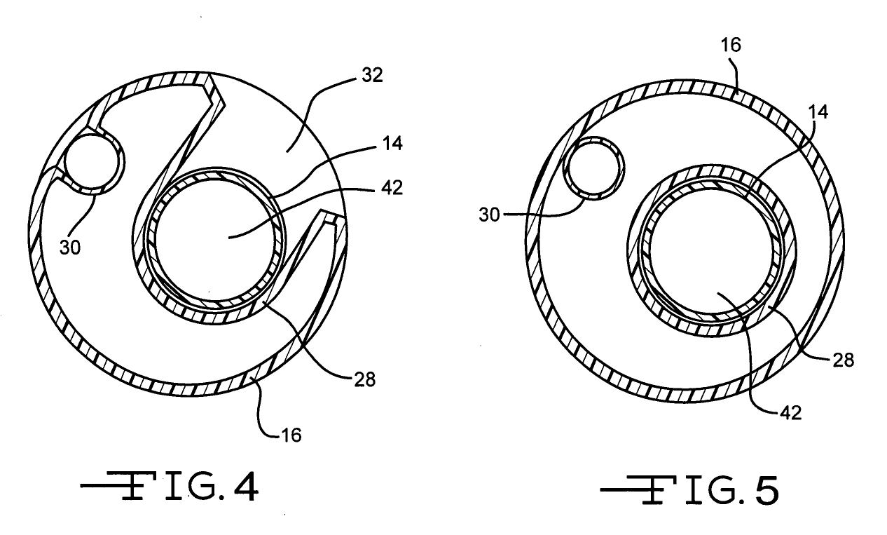

As particularly shown in FIGS. 3 and 4, the primary window 32 comprises spaced apart right and left sides 32A and 32B extending to and meeting with upper and lower edges 32C and 32D. The right and left sides 32A, 32B, which are parallel to each other and to the longitudinal axis of the housing 12, are recessed into the thickness of the sidewall 16. The upper and lower edges 32C, 32D are aligned perpendicular to the longitudinal axis of the housing 12.

The biopsy rod 14 comprises a handle 36 supported at a proximal end 38 opposite a curved radiused distal end 40. The biopsy rod 14 is made of a similar material as the housing and has a circular cross-section perpendicular to its length that is sized to reside in a snug but sliding relationship in the main housing passage 28. The biopsy rod 14 has a length greater than that of the dual passage housing 12 so that when its distal end 40 resides in the distal end 20 of the housing 12, the rod handle 36 extends beyond the open proximal housing end 18. An internal rod passage 42 communicates from the proximal end at the handle 36 to a cutting recess 44 adjacent to the distal end 40. The cutting recess 44 has a length along the biopsy rod 14 that substantially matches the length of the housing window 32.

In use, the biopsy rod 14 is moved into the main passage 28 of the housing 16 until its radiused end 40 seats in the rounded terminus 44 of the main passage 28. Then to perform a biopsy, the needle device 10 comprising the biopsy rod 14 received in the dual-passage housing 12 is moved into a body until the window 32 is located at a tissue of interest. As this movement occurs, the biopsy rod 14 is positioned in the main channel 28 with a portion of its curved sidewall opposite the cutting recess 44 closing the window 32. Then, the biopsy rod 14 is rotated until the cutting recess 44 is aligned with the window 32. This allows a sample of body tissue to fill into the window 32 by the natural pressure of the body. One of its opposed cutting edges 44A, 44B cuts a sample of the body tissue as further rotational movement of the rod continues until the window 32 is again closed.

If bleeding should occur at the biopsy site or if the surgeon otherwise determines that it is necessary, a medicine or drug can be delivered to the biopsy site through the secondary opening 34 in the dual-passage housing 12. As shown in FIG. 1, the syringe 22 including a flexible polymeric conduit 48 is connected to the secondary passage 30 at the open proximal end 18 of the housing 12 to deliver a medicine therein which is administered to the biopsy site through the opening 34. The medicine can be gravity fed or allowed to drip down the secondary passage to the secondary opening. Alternatively, the plunger on the syringe 22 can be manipulated to deliver a relatively large amount of medicine to the biopsy site in a short period of time.

In another embodiment, a drug delivery tube 50 (FIG. 2) is connected to the housing 12 spaced from the proximal open end 18 thereof. The tube 50 is preferably of the same material as the housing 12, such as titanium or stainless steel, and is welded or otherwise secured to the sidewall 16 at the proximal end of the secondary passage 30. Alternatively, the tube 50 can be of a flexible polymeric material. In any event, the tube in fluid flow communication with the secondary passage 30 through the housing sidewall 16 serves to deliver medicine to the biopsy site through the secondary opening 34. This delivery is by gravity feed. However, it is within the scope of the present invention that the syringe 22 can be connected to the secondary passage 30 through the housing sidewall 16 as shown in FIG. 2 and that the tube 50 can be connected to the proximal open end 18 of the housing shown in FIG. 1.

Another embodiment of the present invention comprises any one of a variety of surgical tools, for example a hypodermic needle device 100. The hypodermic needle device 100 comprises a tool rod 102 provided with side-by-side dual-passages 104 and 106. The main passage 104 is in fluid flow communication with the hypodermic needle cutting edge 108 while the secondary passage 106 communicates with the secondary opening 110. The tool rod 102 is preferably of titanium or stainless steel or of a biocompatible polymeric material.

The tool rod 102 is locked inside a sheath 112 for moving to the surgical site through an elongate and flexible cannula (not shown). The tool rod 102 and sheath 112 are each provided with J-shaped channels 114, 116 that cooperate with locking pins supported by the sheath and cannula, respectively (only locking pin 118 on the sheath 112 is shown). The cooperating locking pins/J-shaped channels serve as structures for locking and unlocking the hypodermic needle 108 inside the sheath 112 and the sheathed tool inside the cannula.

To perform a surgical procedure, the tool rod 102 supporting the hypodermic needle 108 is locked inside the sheath 112 by manipulating the tool rod until the sheath's locking pin 118 resides in a terminus of the tool rod's J-shaped channel 114. The hypodermic needle 108 locked inside the sheath 112 is then moved through the cannula to a location proximate a surgical site. The sheath 112 helps prevent the hypodermic needle 108 from cutting the inside of the cannula during this movement.

The sheath 112 is then locked inside the cannula by manipulating the tool rod/sheath assembly until the cannula's locking pin resides in a terminus of the sheath's J-shaped channel 116. Next, the tool rod 102 is manipulated in a reverse manner to free it from the sheath 112 by unlocking the sheath's locking pin 118 from the tool rod's J-shaped channel 114. The tool rod 102 in now moved out of the sheath 112 and the cannula to enable the surgeon to administer a drug or medicine through the primary passage 104 to the surgical site through the hypodermic needle 108. If bleeding should occur at this site or if the surgeon otherwise determines that it is necessary, another medicine or drug can be delivered through the secondary passage 106 to the site through the secondary opening 110 in the dual-passage tool rod 102.

Once that is completed, the tool rod 102 is retracted and locked inside the sheath 112, which, in turn, is locked inside the cannula. The tool rod/sheath assembly is then unlocked from the cannula and removed there from. Another surgical tool, such as a scalpel at the end of another tool rod is then locked inside the sheath. A scalpel negates the need for the primary passage. However, the secondary passage 106 and its associated opening 110 are still needed for administering a medicine to the site being worked on by the scalpel without removing the scalpel from the body. This assembly is then moved into the cannula to a position adjacent to the surgical site, and the procedure is repeated. This continues until the surgical procedure is completed.

For a more complete discussion of the functioning of a J-shaped channel/locking pin surgical device according to this embodiment of the present invention, reference is made to U.S. Pat. No. 7,041,050 to Ronald. This patent is assigned to the assignee of the present invention and incorporated herein by reference.

The present invention is not intended to be limited by the described embodiments, but by the herein appended claims.

What is claimed is:

1. A surgical device for performing a surgical procedure, which comprises:
a) a housing comprising a housing sidewall extending along a longitudinal axis from an open proximal end to a distal end thereof;
b) a primary passage in the housing extending from the open proximal housing end to a distal housing end at a needle cutting edge;
c) a secondary passage in the housing extending from the open proximal housing end to a distal opening in the housing sidewall adjacent to the distal housing end;
d) wherein the primary passage enables a surgeon to administer a first drug or medicine to a surgical site during the surgical procedure; and
e) wherein the secondary passage enables the surgeon to administer a second drug or medicine to the surgical site.

2. The surgical device of claim 1 the secondary passage permits the second drug or medicine to be administered substantially at the same time that the first drug or medicine is being administered.

3. The surgical device of claim 1 the secondary passage permits the second drug or medicine to be administered some time after the first drug or medicine has been administered.

4. The surgical device of claim 1 wherein the housing is made of a material selected from the group consisting of titanium, stainless steel, and a polymeric material.

5. The surgical device of claim 1 wherein the distal opening of the secondary passage is at a location either proximal to or directly opposite the needle cutting edge.

6. The surgical device of claim 1 including a drug delivery tube in fluid flow communication with at least one of the primary passage and the secondary passage.

7. The surgical device of claim 1 including a syringe in fluid flow communication with at least one of the primary passage and the secondary passage.

8. A surgical device for performing a surgical procedure, which comprises:
a) a housing comprising a housing sidewall extending along a longitudinal axis from a proximal end to a distal end thereof;
b) a surgical tool providing a cutting edge at the distal end of the housing;
c) a primary passage in the housing extending from an open proximal housing end to a distal opening at the distal housing end adjacent to the cutting edge;
d) wherein the cutting edge enables a surgeon to perform a surgical procedure at a surgical site; and
e) wherein the primary passage enables the surgeon to administer a drug or medicine to the surgical site.

9. The surgical device of claim 8 the primary passage permits the drug or medicine to be administered substantially at the same time that the surgeon is performing the surgical procedure.

10. The surgical device of claim 8 the primary passage permits the drug or medicine to be administered some time after the surgeon has performed the surgical procedure.

11. The surgical device of claim 8 wherein the housing is made of a material selected from the group consisting of titanium, stainless steel, and a polymeric material.

12. The surgical device of claim 8 wherein the distal opening of the primary passage is either proximal to or directly opposite the cutting edge of the surgical tool.

13. The surgical device of claim 8 including a drug delivery tube in fluid flow communication with the primary passage.

14. The surgical device of claim 8 including a syringe in fluid flow communication with the primary passage.

15. A method for performing a surgical procedure, comprising the steps of:
a) providing a surgical device comprising a housing having a housing sidewall extending along a longitudinal axis from an open proximal end to a distal end thereof; a primary passage in the housing extending from the open proximal housing end to a distal housing end at a needle cutting edge; and a secondary passage in the housing extending from the open proximal housing end to a distal opening in the housing sidewall adjacent to the distal housing end;
b) performing a surgical procedure at a surgical site;
c) administering a first drug or medicine to the surgical site through the primary passage during the surgical procedure; and
e) further administering a second drug or medicine to the surgical site through the secondary passage.

16. The method of claim 15 including administering the second drug or medicine to the surgical site either substantially at the same time as administering the first drug or medicine or some time after administering the first drug or medicine.

17. The method of claim 15 including providing the distal opening of the secondary passage at a location selected from one of proximal to, distal to, and directly opposite with respect to the needle cutting edge.

18. The method of claim 15 including providing a drug delivery tube in fluid flow communication with at least one of the primary passage and the secondary passage.

19. The method of claim 15 including providing a syringe in fluid flow communication with at least one of the primary passage and the secondary passage.

20. A method for performing a surgical procedure, comprising the steps of:
a) providing a surgical device comprising a housing having a housing sidewall extending along a longitudinal axis from a proximal end to a distal end thereof; a surgical tool providing a cutting edge at the distal end of the housing; a primary passage in the housing extending from an open proximal housing end to a distal opening at the distal housing end adjacent to the cutting edge;
b) performing a surgical procedure at a surgical site using the cutting edge; and
c) administering a drug or medicine to the surgical site through the primary passage either substantially at the same time as performing the surgical procedure using the cutting edge or some time thereafter.
